(19) United States
(12) Patent Application Publication
(10) Pub. No.: US 2011/0046571 A1
(43) Pub. Date: Feb. 24, 2011

(54) Absorbing and Saturation Detection Pad and Methods Thereof

(76) Inventor: Joshua Waldhorn, Kfar Shmariyahu (IL)

Correspondence Address: The Law Office of Michael E. Kondoudis, 888 16th Street, N.W., Suite 800, Washington, DC, 20006, US

(21) Appl. No.: 12/938,000

(22) PCT Filed: Apr. 15, 2008
(86) PCT No.: PCT/IL08/00513

§ 371 Date: Oct. 15, 2010

(51) Int. Cl. A61F 13/42; A61L 15/46; A61M 31/00

(52) U.S. Cl. 604/246; 604/361; 604/359; 604/286

(57) ABSTRACT

The present invention provides an absorbing pad for indicating saturation. The pad comprises inter alia from at least one absorbent body, at least one saturation sensor embedded within said absorbent body and at least one saturation indicator connected to said saturation sensor, embedded within said absorbent body wherein said indicator is adapted to provide information to the user as to the degree of saturation of said pad without removing the pad from its application.

### FIELD OF THE INVENTION

This invention relates generally to absorbent pads and more particularly to tampons comprising a saturation detection system that signals the user that the tampon is approaching absorbent capacity (or full) and it is time to change the tampon.

### BACKGROUND OF THE INVENTION

Menstrual pads are available in a wide variety of sizes, shapes, and brands. There are maxi pads for heavy days, and mini pads for light days. Some pads are thick and some are thin. Some even conform to the style of panties you wear. And some have “wings” that fit over your panties to hold them in place. Anyone who's every given birth probably can't forget the largest pads—the maternity pads.

Tampons are small rolls of absorbent material that are inserted into the vagina to absorb menstrual flow. They come in different absorbencies. Most tampons come in smooth applicators which make them easy to insert; you throw away the applicator after you've put in the tampon. You remove tampons by pulling on the attached string, which hangs outside the body. Tampons entered the American market in the late 1920's or 30's, however tampon-like materials have been used by women probably for thousands of years. Many of the first commercial tampons did not have an applicator, and one did not have a string. Tampax was the first tampon to have an applicator in 1936. If you use a tampon, you should use the minimum absorbency necessary to manage your menstrual flow on a given day.

Today women have a wide choice of brands of tampons available. Some have cardboard applicators, some plastic, and others no applicator. Some tampons contain deodorants to help reduce menstrual odors. There is much controversy about the safety of tampons and their possible connection to women's health conditions such as endometriosis and toxic shock syndrome (TSS). TSS is caused by staphylococci in the vagina that enters the bloodstream. Although scientists have recognized a connection between tampons and TSS, the exact connection remains unclear. The link is not clearly understood. However, tampon research highlights three high risk factors: high absorbency, continuous use and low body immunity.
Absorbency: the higher the absorbency the higher the risk; the lower the absorbency the lower the risk. That is why a woman should always use the lowest absorbency tampon for her menstrual flow. It also accounts for the high number of deaths due to super-absorbent tampons in 1980.
Continuous use: women should not use tampons continuously during a period. It is recommended that the most convenient time to break the continuous use is at night, by using a sanitary towel/pad.
Low immunity: this is the factor that you cannot control as it may vary from time to time. It is generally understood that immunity improves with age therefore girls are at a higher risk that older women.

The U.S. FDA suggests the following guidelines for decreasing the risk of contracting TSS when using tampons:
Follow package directions for insertion
Choose the lowest absorbency for your flow
Change your tampon at least every 4 to 8 hours
Consider alternating disposable or cloth pads with tampons
Avoid tampon usage overnight when sleeping
Know the warning signs of toxic shock syndrome

Women who enjoy the convenience of tampons but who are concerned about possible health risks can find all natural, organic, cotton tampons on several websites, as well as at their local organic market.

In Western culture, most women choose to use either tampons or disposable sanitary napkins to handle their menstrual flow. Other choices include reusable menstrual cups, disposable menstrual cups, cloth menstrual pads, using a diaphragm as a menstrual cup, or sea sponge tampons.

Prior to the development of tampons, Western women generally resorted to reusable cloth rags. These would be soaked in a diaper pail after use. Rags continue to be used by women in some Third-World countries today, including much of Africa, out of affordability and distribution problems associated with other methods.

### Tampon Absorbency Ratings

What all currently available tampons have in common is an absorbency rating system to help you determine which tampon is right for your flow. Some companies sell boxes of tampons with various sizes in one box so that you can use the smaller ones on your lighter days and the more absorbent tampons on your heaviest days.
Junior: The junior tampon will hold up to 6 grams of menstrual fluid.
Regular: Regular absorbency tampons hold between 6 and 9 grams of menstrual fluid.
Super: These super tampons have an absorbency rating of 9 to 12 grams of menstrual fluid.
Super plus: Super plus tampons are for your heaviest days and absorb from 12 to 15 grams of menstrual fluid.

1 gram of menstrual fluid equals about ¼ teaspoon.

Tampons should be changed every 4 to 6 hours. If your tampon doesn't need changing in 4 to 6 hours, you are using a tampon with too high an absorbency rating and should switch to a lower absorbency tampon. Other signs you are using a tampon that is too absorbent include:
Difficult removal.
Dry vagina.
Tampon shredding upon removal.

Properly inserted tampons are comfortable to wear and do not cause pain or other irritation. You should not be able to feel your tampon when it is inserted correctly. If you can feel your tampon in your vagina then you will need to reinsert it deeper. Tampons are a great choice for women who are physically active. They do not interfere with exercise or swimming. You should not have an odor when you are wearing a tampon—this could be a sign of infection. An odor can also be a sign that you have forgotten to remove a tampon. Tampons can be safely used by women and girls of all ages. If your daughter feels comfortable using tampons, she can use them beginning with her first periods.

Tampons are designed to contain a particular amount of menstrual fluid. The amount of menstrual fluid absorbed by a tampon can vary depending on absorbency levels. For example, in the United States, tampon absorbency can range from less than 6 grams, Junior absorbency, to 15-18 grams, Ultra absorbency (the numbers are taken from U.S. Pat. No. 7,214,848).

Present-day users must remove the tampon in order to ascertain whether a tampon has reached its absorbent capacity, resulting in the destruction of the tampon, as most women are reluctant to reinsert the tampon into the vagina. Generally a user will remove a tampon before it has reached its absorbent capacity in order to prevent an accident wherein the absorbent capacity of the tampon is exceeded. Once the absorbent capacity is exceeded, the excess menses flows out from the vagina to soil the user's clothing.

In tampons as presently known, a criteria frequently used for removing a tampon is time elapsed since insertion. The time elapsed criteria for changing tampons is not satisfactory for several reasons, e.g., the menstrual flow rate varies throughout the menstruating period and much adsorbent capacity of tampons is wasted due to the tendency to change before an accident occurs.

The flow variation throughout the “period” causes problems as to how long to wear a tampon because a user cannot establish a definite time period for which the absorbent capacity within a tampon is sufficient. Therefore, the users are in a quandary as to how long to wear specific tampons during days of heavy flow as contrasted to day of light flow.

Because a correlation between tampon performance during light flow versus heavy flow is difficult to make by the user, users would rather be safe than sorry (and be under constant tension and fear), thus the user frequently removes a tampon before the absorbent capacity of the tampon has been reached and wastes much of the product she had purchased. Due to that, money, time and thoughts are wasted.

Therefore, in order to provide a longer wearing time, tampons have been made larger and with different materials to obtain higher absorbencies, often resulting in product claims that a user would not have to change the tampon as often. Still, that solution did not approach the problem of fully using the absorbent capacity within a tampon and the user would still waste a portion of the tampon absorbent capacity, as most users are not willing to risk having an accident. In rare cases, should a user forget to remove and left the tampon in for an extended period of time, a life threatening infection may be develop.

Moreover, wearing a larger tampon or high absorbency tampon can lead to discomfort and other problems as well. Women will sometimes wear a larger absorbency tampon due to the fear of tampon failure, especially if she is uncertain how often she will be able to access privacy in order to change the tampon. If the tampon is unsaturated, there may be drying of the vaginal wall, which may cause discomfort upon the tampon's removal.

These problems associated with the use of tampons were recognized and here are some of the solutions that were proposed:

U.S. Pat. No. 3,794,024 to Kolx discloses an indicator in contact with the absorbent body of a catamenial device. The indicator “reads” the saturation of the absorbent body and translates the saturation into a signal which can be sensed without removing the catamenial device. Change in temperature (heat generation or consumption by dissolving material) or color formation are used as indicators.

International publication number WO07044162A1 given to Olson describes changes in the temperature as the indicators.

However, these approaches suffer limitations. The amount of heat generated or consumed depends upon the reaction rate. If it is slow, heat dissipation will prevent sensing the signal.

Often visual indications (i.e. color) are also used as an indicator. Visual indications of wet diapers are disclosed in U.S. Pat. No. 3,952,746 to Summers; and U.S. Pat. No. 4,022,211 to Timmons;

That approach, as the first one, suffers limitations. By using color indication, an intimate inspection is requires by the user.

U.S. Pat. Nos. 6,348,640 or 5,904,671 to Navot which discloses a tampons which contains a radio frequency identification device including a transmitter, a conductive saturation sensor and a remote reporting device including a receiver for receiving a radio signal concerning the saturation of the absorbent body from the radio frequency identification device and a reporting mechanism for reporting a user of the saturation of the absorbent body. The reporting mechanism acquire many forms, such as a liquid crystal display (LCD), light emitting diode (LED), vibrator or a sound alarm.

However, in that approach the reporting mechanism (whatever it is) is external to the vagina, and therefore very complicated.

Thus, there is a long felt need for a saturation detection system that is internal to the vagina and signals the user that the capacity of pad is reached without the above limitations.

### SUMMARY OF THE INVENTION

The present invention provides an absorbing pad for indicating saturation, comprising; at least one absorbent body; at least one saturation sensor embedded within said absorbent body; and, at least one saturation indicator connected to said saturation sensor, embedded within said absorbent body; wherein said indicator is adapted to provide information to the user as to the degree of saturation of said pad without removing said pad from its application.

It is another object of the present invention to provide the absorbing pad as defined above, wherein said saturation indicator is visible to the user whilst said pad remains applied.

It is another object of the present invention to provide the absorbing pad as defined above, additionally comprising at least one sensible alert mechanism.

It is another object of the present invention to provide the absorbing pad as defined above, additionally comprising at least one audible alert mechanism.

It is another object of the present invention to provide the absorbing pad as defined above, additionally comprising at least one means for transmitting the saturation status of said pad to a wireless device.

It is another object of the present invention to provide the absorbing pad as defined above, additionally comprising at least one means for receiving said transmission embedded in a device selected from a group including: the pad's container or wrapping, a ring, a bangle, a watch, spectacles, a computer, a pda, an earring, and a phone.

It is another object of the present invention to provide the absorbing pad as defined above, additionally comprising at least one means for fastening said pad selected from a group including: string, lace, strap, harness, clasp, Velcro, adhesive, electro-static and suction.

It is another object of the present invention to provide the absorbing pad as defined above, wherein said pad is selected from a group including: tampons, surgical pads, wound dressings and bandages.

It is another object of the present invention to provide the absorbing pad as defined above, wherein the general structure of said pad is selected from a group including flat and voluminous structure.

It is another object of the present invention to provide the absorbing pad as defined above, wherein the surface area of said pad is enlarged by a plurality of surface enhancing absorbent receptors.

It is another object of the present invention to provide the absorbing pad as defined above, wherein the absorbent material is swirled or weaved so as to retain its volume once saturated.

It is another object of the present invention to provide the absorbing pad as defined above, wherein said pad is utilized for human beings or animals.

It is another object of the present invention to provide the absorbing pad as defined above, wherein said pad is used in a body cavity to absorb non menstrual fluids.

It is another object of the present invention to provide the absorbing pad as defined above, wherein said pad is used to absorb liquids from the lungs of a human being or animals.

It is another object of the present invention to provide the absorbing pad as defined above, wherein the surface of said pad is smooth.

It is another object of the present invention to provide the absorbing pad as defined above, wherein the surface of said pad is perforated.

It is another object of the present invention to provide the absorbing pad as defined above, wherein the surface of said pad is scored.

It is another object of the present invention to provide the absorbing pad as defined above, wherein the absorbent body is composed of hygroscopic materials selected from a group comprising of acetobacteria, cotton wool, cotton, minerals and wood with high percentage of cellulose, clay, cement, rorisite or synthetic fibers.

It is another object of the present invention to provide the absorbing pad as defined above, wherein the hygroscopic materials composing the absorbent body will give qualities of not inflating yet sealing so that it would be able to prevent an excess of fluids from flowing out of the body cavity or wound.

It is another object of the present invention to provide the absorbing pad as defined above, wherein the hygroscopic materials composing the absorbent body provide for a degree of elasticity.

It is another object of the present invention to provide the absorbing pad as defined above, wherein the absorbent body is treated on its outer side with an oily substance in order to provide for an ease of insertion and easy removal.

It is another object of the present invention to provide the absorbing pad as defined above, wherein the reporting mechanism includes more than one reporting mode and additionally comprises a mode selector that enable the selection of a desired reporting mode.

It is another object of the present invention to provide the absorbing pad as defined above, additionally comprising counter odor agents embedded within said absorbent material.

It is another object of the present invention to provide the absorbing pad as defined above, additionally comprising a means for extracting said pad selected from a group including: a string, a lace, a strap and an extraction ring.

It is another object of the present invention to provide the absorbing pad as defined above, additionally comprising a disposable means that retains the hygienic condition of said pad.

It is another object of the present invention to provide the absorbing pad as defined above, additionally comprising an applicator means for guiding said pad into a body cavity.

It is another object of the present invention to provide the absorbing pad as defined above, wherein said applicator means is disposable.

It is another object of the present invention to provide an absorbing pad for indicating saturation, comprising;
i. at least one absorbent body; and,
ii. at least one saturation indicator, including at least one saturation sensor and at least one reporting mechanism; said reporting mechanism is provided with means to give a sensible indication only after the capacity of said pad is reached,
wherein said pad is inserted as is and wholly into a body cavity.

It is another object of the present invention to provide the absorbing pad as defined above, wherein said pad is used for surgeries or in any kind of wounds.

It is another object of the present invention to provide the absorbing pad as defined above, wherein said pad additionally comprising a container for accommodating medicament selected from a group consisting of antibiotic agents for TSS treatment.

It is another object of the present invention to provide the absorbing pad as defined above, additionally comprising an electrically or chemically triggered release valve that releases said medication at a rate that is correlated with the degree of saturation sensed by said interconnected saturation sensor.

It is another object of the present invention to provide the absorbing pad as defined above, wherein said pad is coated with self-dissolving materials selected from a group consisting of Polydioxanone (PDO), Polycaprolactone (PCL), Polylactic acid (PLA), Polyglycolic acid (PGA), Adipic acid, PEG and glutamic acid.

It is another object of the present invention to provide the absorbing pad as defined above, wherein said pad is coated with biocompatible materials.

It is another object of the present invention to provide a method for signaling the user when the absorbing capacity of an absorbing pad has been reached. The method comprising steps selected inter alia from:
a. obtaining an absorbing pad for indicating saturation, comprising:
   i. at least one absorbent body;
   ii. at least one saturation sensor embedded within said absorbent body; and,
   iii. at least one saturation indicator connected to said saturation sensor;
b. applying said pad;
c. observing said saturation indicator; and,
d. replacing said pad if said indicator indicates said pad has reached its capacity.

It is another object of the present invention to provide a method for signaling the user when the absorbing capacity of an absorbing pad has been reached. The method comprising steps selected inter alia from:
a. obtaining an absorbing pad for indicating saturation, comprising:
   i. at least one absorbent body;
   ii. at least one saturation sensor embedded within said absorbent body; and
   iii. at least one saturation indicator connected to said saturation sensor;
   iv. at least one sensible alerting mechanism;
b. applying said pad;
c. sensing said sensible alerting mechanism; and,
d. replacing said pad if said indicator indicates said pad has reached its capacity.

It is another object of the present invention to provide a method for signaling the user when the absorbing capacity of an absorbing pad has been reached. The method comprising steps selected inter alia from:
a. obtaining an absorbing pad for indicating saturation, comprising:
   i. at least one absorbent body;
   ii. at least one saturation sensor embedded within said absorbent body;
   iii. at least one saturation indicator connected to said saturation sensor;
   iv. at least one transmitting means embedded in said pad; and,
   v. at least one transmission receiving means;
b. applying said pad;
c. transmitting saturation status;
d. receiving said saturation status; and,
e. replacing said pad if said indicator indicates said pad has reached its capacity.

It is another object of the present invention to provide a method for signaling the user when the absorbing capacity of an absorbing pad has been reached. The method comprising steps selected inter alia from:
a. providing an absorbing pad having means to signal said user by giving a sensible indication after the capacity of said pad is reached,
b. applying said pad,
whereby said pad can be extracted after reaching its full capacity.

It is another object of the present invention to provide a method for signaling the user when the absorbing capacity of an absorbing pad has been reached. The method comprising steps selected inter alia from:
a. obtaining an absorbing pad for indicating saturation, comprising:
   i. at least one absorbent body;
   ii. at least one saturation sensor embedded within said absorbent body; and,
   iii. at least one saturation indicator connected to said saturation sensor;
   iv. at least one sensible alerting mechanism;
b. applying said pad;
c. sensing said sensible alerting mechanism;
d. observing said saturation indicator; and,
e. replacing said pad if said indicator indicates said pad has reached its capacity

It is another object of the present invention to provide a method for signaling the user when the absorbing capacity of an absorbing pad has been reached. The method comprising steps selected inter alia from:
a. obtaining an absorbing pad for indicating saturation, comprising:
   i. at least one absorbent body;
   ii. at least one saturation sensor embedded within said absorbent body;
   iii. at least one saturation indicator connected to said saturation sensor;
   iv. at least one transmitting means embedded in said pad; and,
   v. at least one transmission receiving means;
b. applying said pad;
c. transmitting saturation status;
d. receiving said saturation status;
e. observing said saturation indicator; and,
f. replacing said pad if said indicator indicates said pad has reached its capacity.

### BRIEF DESCRIPTION OF THE FIGURES

In order to understand the invention and to see how it may be carried out in practice, a preferred embodiment will now be described, by way of non-limiting example only, with reference to accompanying drawing in which:

FIG. 1 is a schematic illustration of an absorbing pad with a plurality of surface-face increasing absorption receptors, and additionally contains a built-in absorbent capacity indicator window that is interconnected via electro-chemical activator (not shown) to a visual and motion saturation capacity indicating means.

FIG. 1a is a schematic illustration of an absorbing pad revealing its interior composition that provides for the quality of flexibility, oiliness (so that it is easily injected and extracted), ability of rotation and the quality of not inflating yet sealing so that it would be able to prevent an accident from happening (for example, soiling user's clothing by the excess of menses that flows out from the vagina).

FIG. 1b is a schematic view of a preferred embodiment the present invention in which absorbent material of the pad is structured in essentially spiral manner.

FIG. 1c is a schematic view of a preferred embodiment the present invention in which absorbent material of the pad is structured in essentially longitude stripe manner.

FIG. 2 is a schematic view of the interconnection between a visual indicating means of the capacity status, an electro-chemical activator and a battery-operated motorized vibrating means.

FIG. 3a is a schematic view of an absorbing pad in which the indicating window indicates empty status.

FIG. 3b is a schematic view of an absorbing pad in which the indicating window indicates partially full status.

FIG. 3c is a schematic view of an absorbing pad in which the indicating window indicates almost full status.

FIG. 3d is a schematic view of an absorbing pad in which the indicating window indicates full status.

FIG. 4 is a schematic view of an absorption pad ejected securely in the direction of the body cavity (not shown) through integrated direction safety clips by means of a conveyor mechanism.

FIG. 5 is a schematic view of a preferred embodiment of the present invention wherein the absorption pad is essentially flat and is embedded with a visual and motion capacity indicating system, and additionally fitted with fastening straps.

FIG. 5a-c are a schematic top view of a preferred embodiment of the present invention wherein visual indicators embedded into an essentially flat pad indicate saturation capacity status of the pad.

### DETAILED DESCRIPTION OF THE INVENTION

The present invention is a new observation system that includes a detection system which is assembled in a tampon and be used to provide information concerning, for example, a presence of a change in concentration of one or more specific substances, such as, but not limited to, menses, blood, water, sugars, minerals, ions, salts, proteins, toxins, microorganisms in human being or animals. It can provide information about saturation, pH et cetera. The present invention can be used, especially, to provide the user with information concerning the capacity of the tampon, meaning, if the tampon is about to be full ant it should be removed. An important feature of the present invention is that it diminished the need for constant intimate inspection by the user and that both the detecting and reporting mechanism are inserted into the vagina, lungs, any other body cavity or wound.

Before explaining the figures, it should be understood that the invention is not limited in its application to the details of construction and the arrangement of the components set forth in the following description or illustrated in the drawings. The invention can be carried out in various ways. Also, it should be understood that the invention is not limited in its application to feminine hygiene and can be used in any other body cavity (not only the vagina) and in surgeons and wounds, if needed.

The present invention provides an absorbing pad for indicating saturation, comprising; at least one absorbent body; and, at least one indication device, including at least one saturation sensor and at least one reporting mechanism; said reporting mechanism provided with means to give a sensible indication only after the capacity of said pad is reached, wherein said pad is inserted as is and wholly into a body cavity.

The present invention also provides a method for signaling the user when the absorbing capacity of an absorbing pad has been reached comprising the steps of: providing said pad with means to signal the user by giving a sensible indication after the capacity of said pad is reached, inserting said pad into a body cavity, whereby said pad can be extracted from the body after reaching its full capacity.

The term “absorbent body” refers hereinafter to any absorbent material which can absorb body fluid. One of the options is hygroscopic material such as, but not limited to, acetobacteria, cotton wool, minerals and wood with high percentage of cellulose.

The term “saturation” refers hereinafter to a predetermined relative degree of saturation that is less or equal to the absolute degree of saturation. It further applies to all body fluids (menses, blood, water et cetera). It also refers to sugars, minerals, ions, salts, proteins, toxins and microorganisms.

The term “indication device” refers hereinafter to a device which contains at least one saturation sensor and at least one reporting mechanism. The indication device senses when the capacity of the pad is reached and signals it to the user.

The term “saturation sensor” refers hereinafter to a device which can inform the reporting mechanism that the pad had reached its absorption capacity.

The terms “reporting mechanism” refer hereinafter to device which can give a sensible indication. The reporting mechanism may acquire many forms, such as, but not limited to, vibration, alarm sound, color or light.

The terms “signaling” or “sensible indication” refer hereinafter to the form which the reporting mechanism had acquired. The signal or the sensible indication can be vibration, alarm sound, color, light et cetera.

The term “reaching the pad's capacity” refers hereinafter to the time when menses reach saturation sensor and it becomes conductive.

The term “wound” refers hereinafter to any type of physical trauma wherein the skin is torn, cut or punctured.

The term “body cavity” refers hereinafter to spaces, located between a human being's or an animal's outer covering (epidermis) and the outer lining of the gut cavity, where internal organs develop.

The term “self-dissolving materials” refers hereinafter to materials that are degraded by the body's enzymatic pathways through a reaction against “foreign” material. Some urologists may prefer self-dissolving materials in catheter simply because then they don't have to go necessarily through the procedure of removing them afterwards. Examples of self-dissolving polymers are Polydioxanone (PDO), Polycaprolactone (PCL), Polylactic acid (PLA), Polyglycolic acid (PGA), Adipic acid, PEG and glutamic acid.

The term “Biocompatible materials” refers hereinafter to materials that have the ability to perform with an appropriate host response in a specific application. Biocompatible materials have the quality of not having toxic or injurious effects on biological systems.

The present invention also provides an absorbing pad wherein said pad is used for surgeries or any kind of wounds.

It is yet according to another embodiment of the present invention, wherein said pad is used in body cavity to absorb on menstrual fluids.

It is yet according to another embodiment of the present invention, wherein said pad is utilize for human being or animals.

It is yet according to another embodiment of the present invention, wherein said absorbent body is composed of hygroscopic materials selected from a group comprising of acetobacteria, cotton, cotton wool, minerals and wood with high percentage of cellulose.

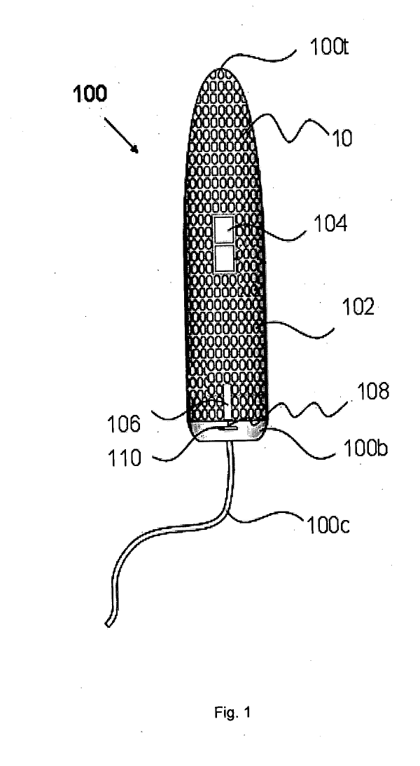

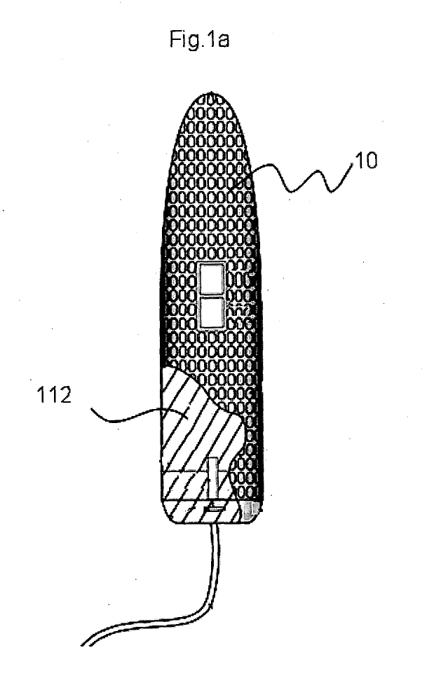

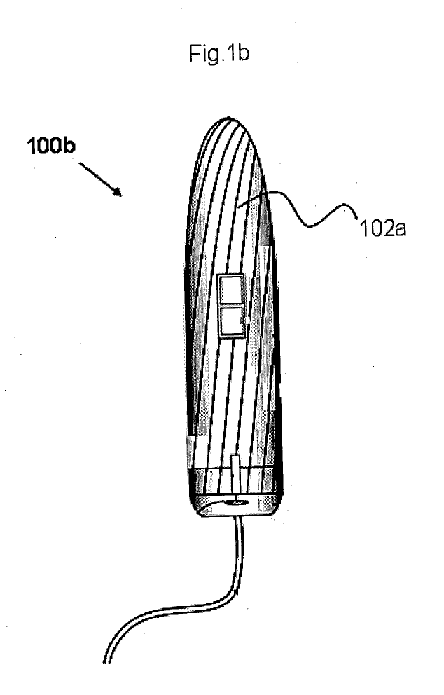

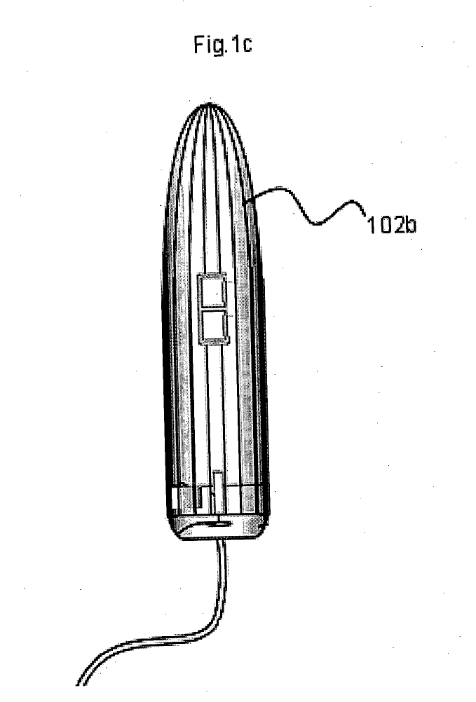

Reference is now made to FIG. 1 which is a schematic view of absorbing pad 100 wherein the body 10 of the absorption pad is to be manually inserted into the body in the direction of tip 100t and removed from the body in the direction of leak prevention base 100b by means of pulling on string 100c. The absorbing is further covered on its outer surface with a plurality of surface-face increasing absorption receptors 102, and additionally contains built-in absorbent capacity indicator window 104 that is interconnected via electro-chemical activator (not shown) to battery operated motor 106 that is in turn interconnected to axle 108 that is interconnected to vibration means 110, which is enclosed in the leak prevention base.

Reference is now made to FIG. 1a which is a schematic view of absorbing pad 100, revealing interior composition 112 of the compressed hygroscopic absorbent material, such as acetobacteria, cotton wool, minerals and wood with high percentage of cellulose. The hygroscopic absorbent materials provide the tampon assembly 100 the quality of flexibility, oiliness (so that it is easily injected and extracted), ability of rotation and the quality of not inflating yet sealing so that it would be able to prevent an accident from happening (for example, soiling user's clothing by the excess of menses that flows out from the vagina). Body 10 is cylindrical, egged shaped and is preferably equipped with a removal string.

Reference is now made to FIG. 1b which is a schematic view of a preferred embodiment 100b of an absorbing pad in which the absorbent material is structured in essentially spiral manner 102a.

Reference is now made to FIG. 1c which is a schematic view of a preferred embodiment 100b of an absorbing pad in which the absorbent material is structured in essentially longitude stripe manner 102b.

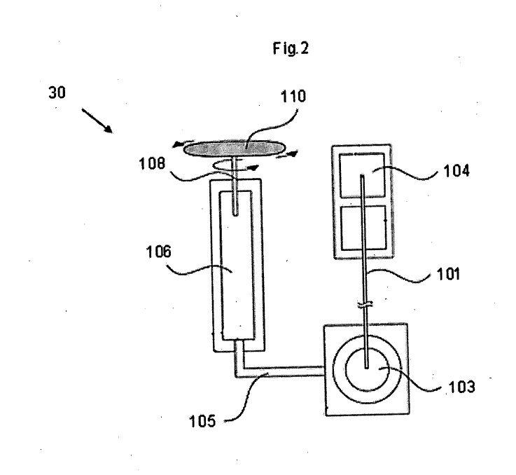

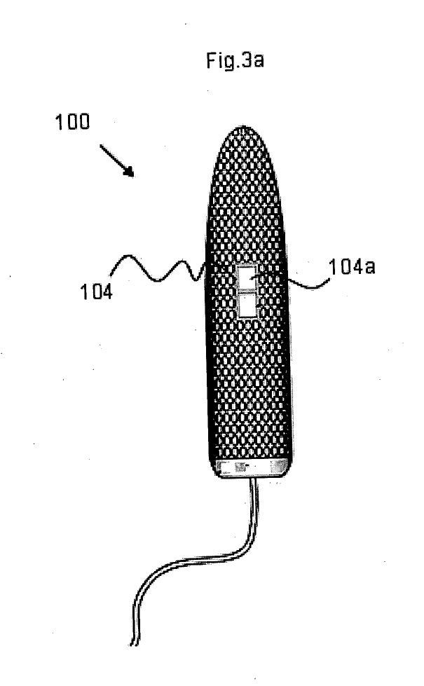

Reference is now made to FIG. 2 which is a schematic view of the interconnection between visual indicating means 104 that conveys its capacity status by means of channel 101 to electro-chemical activator 103 which conveys an electrical signal by means of wire 105 to battery-operated motor 106 that turns axle 108 that causes the vibration of vibration means 110.

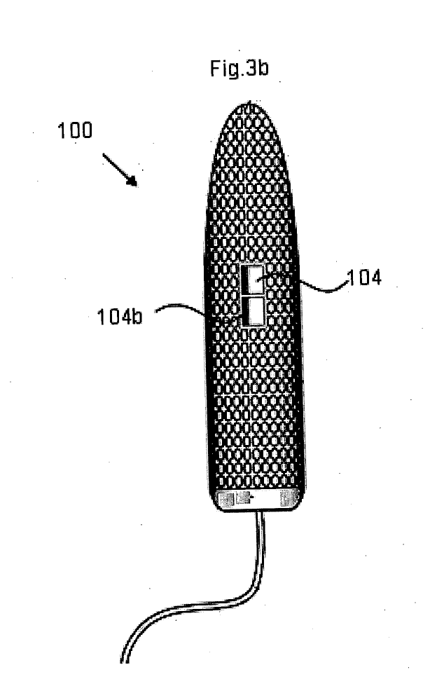

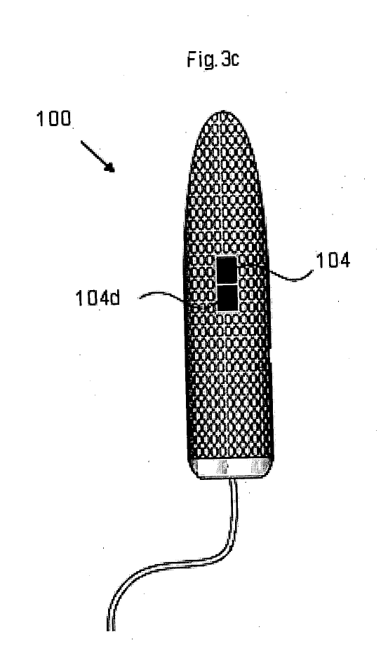

Reference is now made to FIG. 3a which is a schematic view of absorbing pad 100 in which indicating window 104 indicates empty status 104a.

Reference is now made to FIG. 3b which is a schematic view of absorbing pad 100 in which indicating window 104 indicates partially full status 104b.

Reference is now made to FIG. 3c which is a schematic view of absorbing pad 100 in which indicating window 104 indicates almost full status 104c.

Reference is now made to FIG. 3d which is a schematic view of absorbing pad 100 in which indicating window 104 indicates full status 104d.

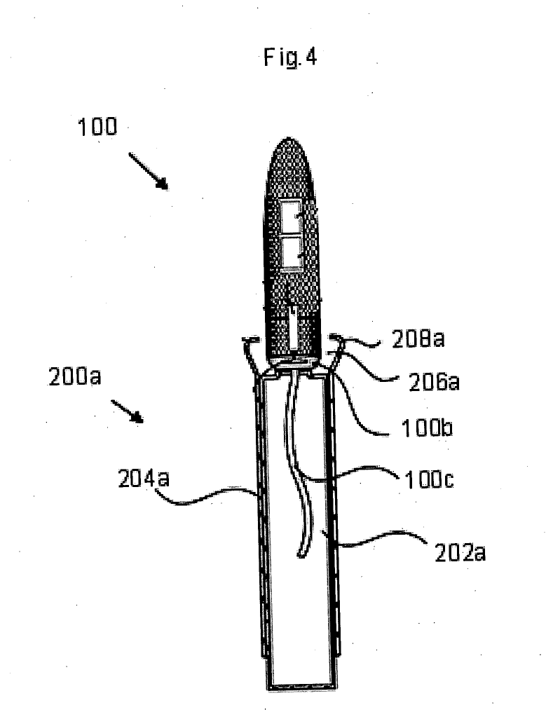

Reference is now made to FIG. 4 depicting a schematic view of absorption pad 100 ejected securely into the body cavity (not shown) through direction safety clips 209a and 208b from conveyor 200a by pushing ejector 200a against base 100b of the absorption pad into the cavity between conveyor walls 204a and 205a that was formerly occupied by the pad.

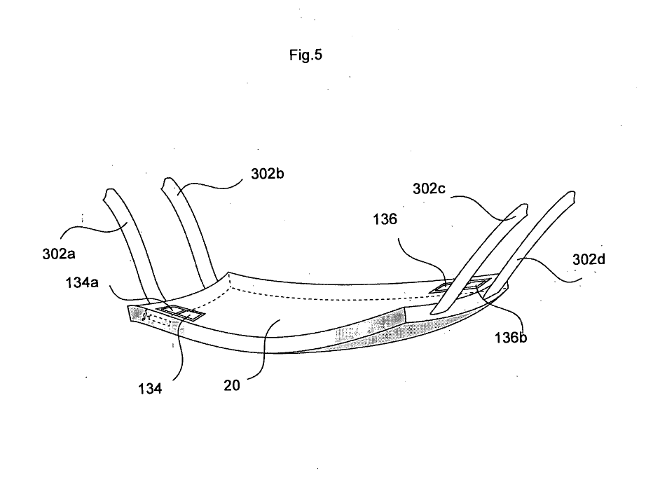

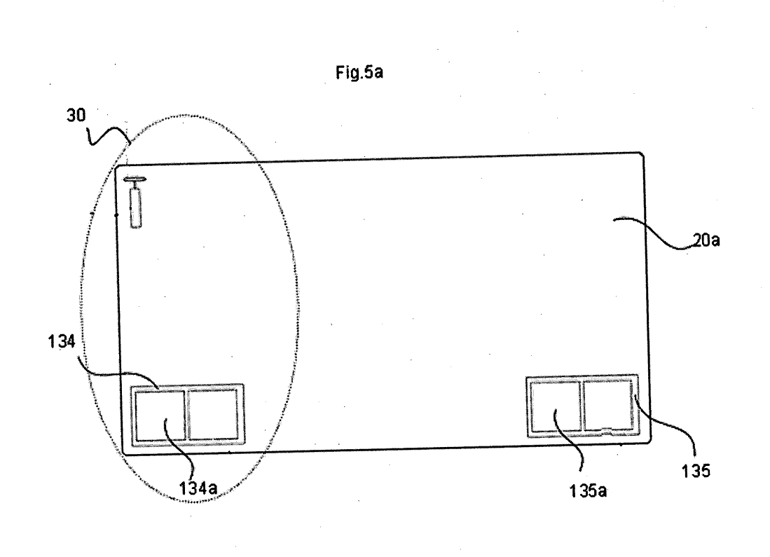

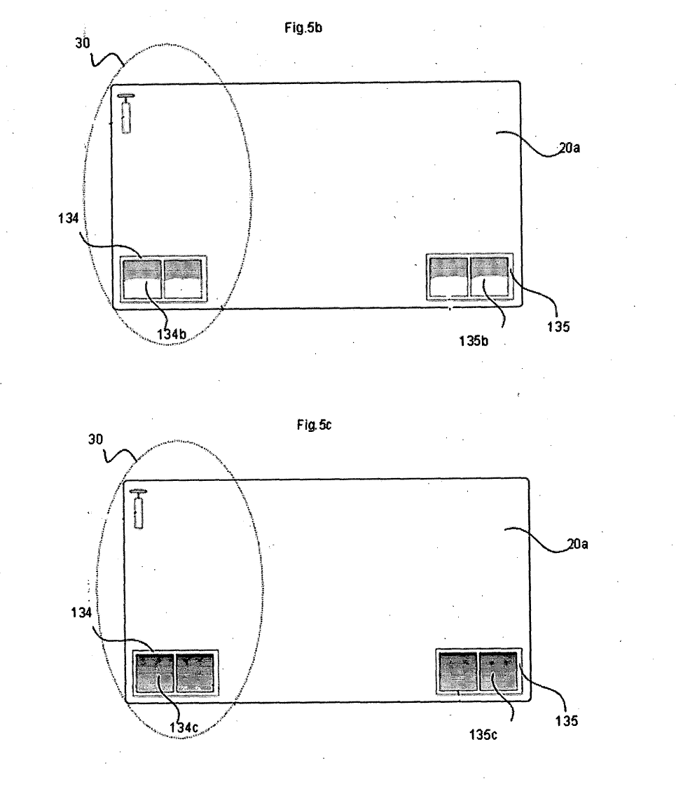

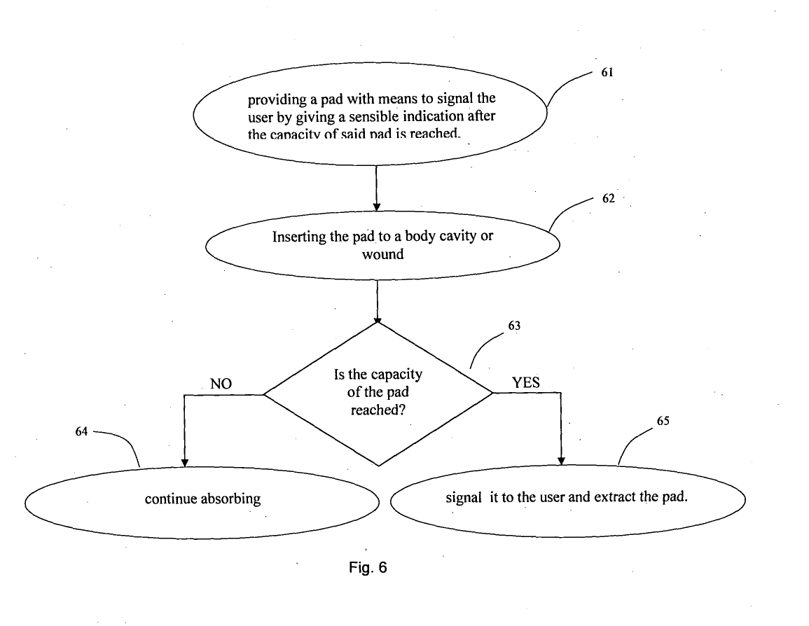

Reference is now made to FIG. 5 depicting a schematic view of a preferred embodiment of the present invention 300 wherein absorption pad 20 is essentially flat and is embedded with visual and motion capacity indicating system 30, and is additionally fitted with fastening straps 302a-d

Reference is now made to FIG. 5a depicting a schematic top view of a preferred embodiment of the present invention 300 wherein visual indicators 134 and 135 indicate empty status 134a,135a.

Reference is now made to FIG. 5b depicting a schematic top view of a preferred embodiment of the present invention 300 wherein visual indicators 134 and 135 indicate partially full status 134b,135b.

Reference is now made to FIG. 5c depicting a schematic top view of a preferred embodiment of the present invention 300 wherein visual indicators 134 and 135 indicate full status 134c,135c.

According to another embodiment of the present invention, the pad additionally comprising a container for accommodating medicament selected from a group consisting of agents for countering staphylococci or otherwise alleviating the threat of toxic shock syndrome (TSS).

According to another embodiment of the present invention, additionally comprising an electrically or chemically-triggered release valve that releases medicament at a rate that is correlated with the degree of saturation sensed by said interconnected saturation sensor.

According to another embodiment of the present invention, the pad is adapted to release medicament by self-degrading materials.

According to another embodiment of the present invention, the pad is coated with biocompatible materials.

Reference is now made to FIG. 6, which illustrate the method for signaling the user when the pad's capacity has been reached. At the first stage (61) a pad with means to signal the user by giving a sensible indication after the capacity of said pad is reached is provided. The next stage (62) is to insert the pad into a body cavity or wound. If said pad had reached its capacity the reporting mechanism is operable and said pad can be extracted (65). Alternatively, if the capacity of the pad is not reached (64) the pad will continue to absorb fluids.

It should be pointed out that the absorbing pad according to any of the above mentioned embodiments is especially adapted to be highly flexible so as to be easily and painlessly inserted, applied, operated and extracted.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An absorbing pad for indicating saturation, comprising;
i. at least one absorbent body;
ii. at least one saturation sensor embedded within said absorbent body; and,
iii. at least one saturation indicator connected to said saturation sensor, embedded within said absorbent body;
wherein said indicator is adapted to provide information to the user as to the degree of saturation of said pad without removing said pad from its application.

2. The absorbing pad according to claim 1, wherein said saturation indicator is visible to the user whilst said pad remains applied.

3. The absorbing pad according to claim 1, additionally comprising at least one sensible alert mechanism.

4. The absorbing pad according to claim 1, additionally comprising at least one audible alert mechanism.

5. The absorbing pad according to claim 1, additionally comprising at least one means for transmitting the saturation status of said pad to a wireless device.

6. The absorbing pad according to claim 5, additionally comprising at least one means for receiving said transmission embedded in a device selected from a group including: the pad's container or wrapping, a ring, a bangle, a watch, spectacles, a computer, a pda, an earring, and a phone.

7. The absorbing pad according to claim 1, additionally comprising at least one means for fastening said pad selected from a group including: string, lace, strap, harness, clasp, Velcro, adhesive, electro-static and suction.

8. The absorbing pad according to claim 1, wherein said pad is selected from a group including: tampons, surgical pads, wound dressings and bandages.

9. The absorbing pad according to claim 1, wherein the general structure of said pad is selected from a group including flat and voluminous structure.

10. The absorbing pad according to claim 1, wherein the surface area of said pad is enlarged by a plurality of surface enhancing absorbent receptors.

11. The absorbing pad according to claim 1, wherein the absorbent material is swirled or weaved so as to retain its volume once saturated.

12. The absorbing pad according to claim 1, wherein said pad is utilized for human beings or animals.

13. The absorbing pad according to claim 1, wherein said pad is used in a body cavity to absorb non menstrual fluids.

14. The absorbing pad according to claim 1, wherein said pad is used to absorb liquids from the lungs of a human being or animals.

15. The absorbing pad according to claim 1, wherein the surface of said pad is smooth.

16. The absorbing pad according to claim 1, wherein the surface of said pad is perforated.

17. The absorbing pad according to claim 1, wherein the surface of said pad is scored.

18. The absorbing pad according to claim 1, wherein the absorbent body is composed of hygroscopic materials selected from a group comprising of acetobacteria, cotton wool, cotton, minerals and wood with high percentage of cellulose, clay, cement, rorisite or synthetic fibers.

19. The absorbing pad according to claim 1, wherein the hygroscopic materials composing the absorbent body will give qualities of not inflating yet sealing so that it would be able to prevent an excess of fluids from flowing out of the body cavity or wound.

20. The absorbing pad according to claim 1, wherein the hygroscopic materials composing the absorbent body provide for a degree of elasticity.

21. The absorbing pad according to claim 1, wherein the absorbent body is treated on its outer side with an oily substance in order to provide for an ease of insertion and easy removal.

22. The absorbing pad according to claim 1, wherein the reporting mechanism includes more than one reporting mode and additionally comprises a mode selector that enable the selection of a desired reporting mode.

23. The absorbing pad according to claim 1, additionally comprising counter odor agents embedded within said absorbent material.

24. The absorbing pad according to claim 1, additionally comprising a means for extracting said pad selected from a group including: a string, a lace, a strap and an extraction ring.

25. The absorbing pad according to claim 1, additionally comprising a disposable means that retains the hygienic condition of said pad.

26. The absorbing pad according to claim 1, additionally comprising an applicator means for guiding said pad into a body cavity.

27. The absorbing pad according to claim 26, wherein said applicator means is disposable.

28. An absorbing pad for indicating saturation, comprising;
i. at least one absorbent body; and,
ii. at least one saturation indicator, including at least one saturation sensor and at least one reporting mechanism; said reporting mechanism is provided with means to give a sensible indication only after the capacity of said pad is reached,
wherein said pad is inserted as is and wholly into a body cavity.

29. The absorbing pad of claim 1 or any of its dependant claims, wherein said pad is used for surgeries or in any kind of wounds.

30. The absorbing pad of claim 1 or any of its dependant claims, wherein said pad additionally comprising a container for accommodating medicament selected from a group consisting of antibiotic agents for TSS treatment.

31. The absorbing pad according to claim 30, additionally comprising an electrically or chemically triggered release valve that releases said medication at a rate that is correlated with the degree of saturation sensed by said interconnected saturation sensor.

32. The absorbing pad of claim 31, wherein said pad is coated with self-dissolving materials selected from a group consisting of Polydioxanone (PDO), Polycaprolactone (PCL), Polylactic acid (PLA), Polyglycolic acid (PGA), Adipic acid, PEG and glutamic acid.

33. The absorbing pad of claim 31, wherein said pad is coated with biocompatible materials.

34. A method for signaling the user when the absorbing capacity of an absorbing pad has been reached; said method comprising steps of:
a. obtaining an absorbing pad for indicating saturation, comprising:
i. at least one absorbent body;
ii. at least one saturation sensor embedded within said absorbent body; and,
iii. at least one saturation indicator connected to said saturation sensor;
b. applying said pad;
c. observing said saturation indicator; and,
d. replacing said pad if said indicator indicates said pad has reached its capacity.

35. A method for signaling the user when the absorbing capacity of an absorbing pad has been reached; said method comprising steps of:
a. obtaining an absorbing pad for indicating saturation, comprising:
i. at least one absorbent body;
ii. at least one saturation sensor embedded within said absorbent body; and
iii. at least one saturation indicator connected to said saturation sensor;
iv. at least one sensible alerting mechanism;
b. applying said pad;
c. sensing said sensible alerting mechanism; and,
d. replacing said pad if said indicator indicates said pad has reached its capacity.

36. A method for signaling the user when the absorbing capacity of an absorbing pad has been reached; said method comprising steps of:
a. obtaining an absorbing pad for indicating saturation, comprising:
i. at least one absorbent body;
ii. at least one saturation sensor embedded within said absorbent body;
iii. at least one saturation indicator connected to said saturation sensor;
iv. at least one transmitting means embedded in said pad; and,
v. at least one transmission receiving means;
b. applying said pad;
c. transmitting saturation status;
d. receiving said saturation status; and,
e. replacing said pad if said indicator indicates said pad has reached its capacity.

37. A method for signaling the user when the absorbing capacity of an absorbing pad has been reached comprising the steps of:
a. providing an absorbing pad having means to signal said user by giving a sensible indication after the capacity of said pad is reached,
b. applying said pad,
whereby said pad can be extracted after reaching its full capacity.

38. A method for signaling the user when the absorbing capacity of an absorbing pad has been reached; said method comprising steps of:
a. obtaining an absorbing pad for indicating saturation, comprising:
i. at least one absorbent body;
ii. at least one saturation sensor embedded within said absorbent body; and
iii. at least one saturation indicator connected to said saturation sensor;
iv. at least one sensible alerting mechanism;
b. applying said pad;
c. sensing said sensible alerting mechanism;
d. observing said saturation indicator; and,
e. replacing said pad if said indicator indicates said pad has reached its capacity;

39. A method for signaling the user when the absorbing capacity of an absorbing pad has been reached; said method comprising steps of:
a. obtaining an absorbing pad for indicating saturation, comprising:
i. at least one absorbent body;
ii. at least one saturation sensor embedded within said absorbent body;
iii. at least one saturation indicator connected to said saturation sensor;
iv. at least one transmitting means embedded in said pad; and,
v. at least one transmission receiving means;
b. applying said pad;
c. transmitting saturation status;
d. receiving said saturation status;
e. observing said saturation indicator; and,
f. replacing said pad if said indicator indicates said pad has reached its capacity.
